(19) United States
(12) Patent Application Publication
(10) Pub. No.: US 2013/0120757 A1
(43) Pub. Date: May 16, 2013

(54) METHODS, SYSTEMS AND APPLICATIONS OF VARIABLE IMAGING DEPTH IN FOURIER DOMAIN OPTICAL COHERENCE TOMOGRAPHY

(71) Applicants: Lingfeng YU, Irvine, CA (US); Matthew J. Everett, Livermore, CA (US); Mary K. Durbin, San Francisco, CA (US); Utkarsh Sharma, San Ramon, CA (US)

(72) Inventors: Lingfeng YU, Irvine, CA (US); Matthew J. Everett, Livermore, CA (US); Mary K. Durbin, San Francisco, CA (US); Utkarsh Sharma, San Ramon, CA (US)

(73) Assignee: Carl Zeiss Meditec, Inc., Dublin, CA (US)

(21) Appl. No.: 13/354,066

(22) Filed: Jan. 19, 2012

Related U.S. Application Data
(60) Provisional application No. 61/435,129, filed on Jan. 21, 2011.
(60) Provisional application No. 61/501,615, filed on Jun. 27, 2011.

(51) Int. Cl. A61B 3/10; G01B 9/02

(52) U.S. Cl. CPC A61B 3/102; G01B 9/02091
USPC 356/479; 351/206

(57) ABSTRACT

Systems, methods and applications for adjusting the imaging depth of a Fourier Domain optical coherence tomography system without impacting the axial resolution of the system are presented. One embodiment of the invention involves changing the sweep rate of a swept-source OCT system while maintaining the same data acquisition rate and spectral bandwidth of the source. Another embodiment involves changing the data acquisition rate of a SS-OCT system while maintaining the same sweep rate over the same spectral bandwidth. Several applications of variable imaging depth in the field of ophthalmic imaging are described.

### PRIORITY

This application claims priority to U.S. Provisional Application Ser. No. 61/435,129, filed Jan. 21, 2011, and U.S. Provisional Application Ser. No. 61/501,615 filed Jun. 27, 2011, the entire contents of which are hereby incorporated by reference.

### TECHNICAL FIELD

One or more embodiments of the present invention relate generally to improvements in Optical Coherence Tomography (OCT) systems, methods, and applications. In particular it is an object of the present invention to enable OCT imaging with adjustable imaging depth, without substantially sacrificing the axial resolution of the system.

### BACKGROUND

Optical coherence tomography is a noninvasive, noncontact imaging modality that uses coherence gating to obtain high-resolution cross-sectional images of tissue microstructure. In Fourier domain OCT (FD-OCT), the interferometric signal between light from a reference and the back-scattered light from a sample point is recorded in the frequency domain rather than the time domain. After a wavelength calibration, a one-dimensional Fourier transform is taken to obtain an A-line spatial distribution of the object scattering potential. The spectral information discrimination in FD-OCT is accomplished either by using a dispersive spectrometer in the detection arm in the case of spectral-domain OCT (SD-OCT) or rapidly tuning a swept laser source in the case of swept-source OCT (SS-OCT).

The axial or depth resolution of the FD-OCT system is determined by the actual spectral width recorded and used for reconstruction. The axial range over which an OCT image is taken (imaging depth, scan depth or imaging range) is determined by the sampling interval or resolution of the optical frequencies recorded by the OCT system. Specifically, in SD-OCT, the spectrometer disperses different wavelengths to the detector elements. The resolution of the optical frequencies and therefore the imaging depth depends on the width of the portion of the spectrum that is measured by a single detector element or pixel.

In some SS-OCT implementations, the swept-source tunes or sweeps the wavelength of the source over time. In this case, the resolution of the optical frequencies depends on a spectral separation of the measuring light at adjacent points in time. The spectral resolution of the measurements will increase with sampling density unless it is limited by the instantaneous linewidth of the laser. For most of the swept-sources, OCT signals acquired with adjacent points separated by uniform (constant) time intervals result in a non-uniform sample distribution in K (wave vector) space. Normally, these optical frequencies are further numerically re-sampled (or interpolated) to get equally K-spaced samples before the Fourier transform is actually taken. This will digitally affect the actual imaging depth in the OCT reconstruction as the imaging depth is now determined by the resolution in wave-numbers (K). In other implementations, Fabry-Pérot interferometers (FPI or etalon) (see for example J. Zhang et al “Swept laser source at 1 um for Fourier domain optical coherence tomography,” Applied Physics Letters 89, 073901 2006) and Mach-Zehnder interferometers (see for example J. Xi et al “Generic real-time uniform K-space sampling method for high-speed swept-source optical coherence tomography,” Optics Express 18(9):9511 2010) can be used to generate external clock signals with uniform K spacing. In this case, the digitizer (or data acquisition system) of the SS-OCT system is running in an “external clock” mode, whereby it takes the external K-clock signals for point by point sampling.

In the past, the use of multiple scan depths in commercially available OCT instruments has been extremely limited. For example commercial FD-OCT instruments such as the Cirrus HD-OCT (Carl Zeiss Meditec, Inc.) have been limited to a single scan depth. Most time-domain retinal imaging OCT instruments (e.g. Stratus, OCT 1, and OCT 2 (Carl Zeiss Meditec, Inc.)) also have a single fixed scan depth. The OCT instrument Visante (Carl Zeiss Meditec, Inc.) has two operating modes. The standard resolution imaging mode provides a broad view of the anterior chamber including the cornea, anterior chamber, iris and both angles with a 16 mm width and 6 mm depth image. The high resolution imaging mode provides a more detailed image of the cornea with a 10 mm width and 3 mm depth. In this case, a tradeoff is made between resolution and scan depth.

Various OCT systems with adjustable resolution and imaging speed in two different imaging modes, i.e., set-up mode and diagnostic mode have been described in the literature. In one case, the extent of the frequency sweep of the light source used to generate the image of the eye during the set-up mode is much smaller than that of the diagnostic mode. In a second case relevant to SD-OCT, the number of detector elements used in set-up mode is about one half or less than the number of detector elements used during the diagnostic mode thereby effectively trading spectral bandwidth for the total time required to read the spectra from the camera. An optional aspect involves narrowing the spectrum of the illumination source in the set-up mode. Another embodiment involves reducing the sweep rate and sweep range simultaneously in the set-up mode. If the rate of sampling and digitization is maintained between the two modes, the samples will be more densely spaced in optical frequency in the set-up mode resulting in a greater depth range. Finally, the spectral width can be adjusted in two imaging modes while the number of spectral samples can be kept constant resulting in a change of the imaging depth allowing structural information to be coarsely resolved in one (set-up mode) measurement or more finely resolved in a second (diagnostic mode) measurement. In all of these cases, the spectral width is dramatically changed from one mode to another, so the axial resolution is greatly sacrificed in one of its two imaging modes.

Systems have been described that include additional hardware or optical components in the OCT system. The effective line width of the detected interference signal can be reduced through the use of periodic optical filters, masks and multiple spectrometers (see for example US Publication No. 2007/0024856). The desired goal is to reduce image fall off, the quality of the image as a function of depth. Embodiments involving two spectrometers or a single spectrometer with an optical switching device are described that effectively reduce the sampling interval and hence increase the imaging depth. While axial resolution is not sacrificed in this case, the requirement of additional customized optical components is a significant disadvantage. An etalon can be used for both spectral filtering of the swept-source and for trigger generation. The free spectral range (FSR) of the etalon defines the separation of the spectral sampling and is a function of the thickness of the etalon, the index of refraction of the material inside the etalon, and the angle of light incidence upon the etalon. One or more of these parameters have to be adjusted to tune the FSR to change the separation of the spectral sampling thus the imaging depth of the SS-OCT system.

In light of these limitations, it is therefore the object of the present invention to improve OCT systems and methods to enable OCT imaging with adjustable imaging depth, without substantially sacrificing the axial resolution of the system. Various aspects of the invention will be described in further detail below. The systems and methods described herein do not require the addition of hardware to the systems and do not require adjustment of the trigger generation interferometer (such as the optical delay of a Mach-Zehnder interferometer (MZI), thickness, refractive index of an etalon (Fabry-Pérot interferometer (FPI)) or the light incident angle to the etalon). As will be explained in greater detail below, this allows an OCT system to have the flexibility to zoom in/out or change the imaging range to address needs of different applications with the same axial resolution. For example, an imaging depth of 2 mm in tissue is normally used for posterior imaging of the eye, while in anterior chamber imaging of the eye, an imaging depth of more than 6 mm in tissue is normally desired. This flexibility in scan range allows one to provide new capabilities in OCT instruments, as will be explained further below. The ability to achieve these capabilities relevant for various imaging applications without a decrease in axial resolution is a significant advantage.

### SUMMARY

It is an object of the present invention to provide an optical coherence tomography system capable of imaging the eye at multiple variable imaging depths without sacrificing the axial resolution of the images. The present invention proposes several ways of adjusting the OCT imaging depth in FD-OCT systems that will be described in detail below.
1) For an SS-OCT system, tuning the swept-source sweep rate to cover the same spectral range or bandwidth, with the same data acquisition rate;
2) For an SS-OCT system, adjusting the data acquisition rate of the digitizer while keeping the swept-source at the same sweep rate, e.g. by, using a frequency multiplier or divider unit to automatically change the sampling frequency of the external clock signal; and
3) In a SD-OCT system, taking (or discarding) signals from every other (or more) detector elements or laterally binning signals of two (or more) adjacent detector elements.

In each case, the axial resolution can be maintained and no additional hardware needs to be included in the system to achieve the change in imaging depth. For SS-OCT systems, both the sweep rate and the data acquisition rate can be adjusted in combination to achieve a desired imaging depth. The present invention is defined by the claims and nothing in this section should be taken as a limitation on those claims.

### BRIEF DESCRIPTION OF THE DRAWINGS

FIG. 1 is a schematic illustration of an optical coherence tomography (OCT) system.

FIG. 2 is a schematic illustration of an embodiment of the present invention in which the sweep rate is adjusted between two OCT imaging modes while the data acquisition rate is maintained.

FIG. 3 is a schematic illustration of a swept-source laser.

FIG. 4 is a schematic illustration of a filter arrangement for tuning a swept-source laser.

FIG. 5 is a schematic illustration of an embodiment of the present invention in which the acquisition rate is adjusted between two OCT imaging modes while the sweep rate of the source is maintained.

FIG. 6(a) is a schematic illustration of an external clock (K-clock) SS-OCT system capable of operation in two different imaging modes with differing imaging depths. The initial external clock signal shown in 6(b) is reduced using a frequency divider unit to produce the external clock signal shown in 6(c).

FIG. 7(a) is a schematic illustration of an external clock (K-clock) SS-OCT system capable of operation in two imaging modes with differing imaging depths. The initial external clock signal shown in 7(b) is input into a Frequency Doubler/Multiplier to generate a second external clock signal of higher frequency shown in 7(c).

FIG. 8 is a schematic illustration of an SS-OCT system with an external clock.

FIG. 9 is a schematic illustration of an embodiment of the invention for an SD-OCT system.

### DETAILED DESCRIPTION

The following definitions are included to provide clarity to the detailed description:

Axial—along the direction of propagation of the OCT imaging light

Axial scan position—Axial position in the tissue at the center of the OCT A-scan

The following parameters are helpful in illustrating the various embodiments of the invention, particularly the swept-source based embodiments:
f_{D}—Duty cycle—fraction of time that the laser has outputs light and data is being acquired
Lₛ (nm/sec)—Laser spectral sweep rate—Rate at which a swept-source laser is being tuned across the spectrum
L_{R} (nm)—Laser spectral sweep range—Spectral range or spectrum of swept-source
Aₛ=f_{D}Lₛ/L_{R} (sec⁻¹)—A-scan rate or # of A-scans per second
Δ_{z}˜1/L_{R} (mm⁻¹)—Axial resolution is defined by inverse of laser spectral sweep range
R (pts/sec)—Data acquisition rate—Rate at which data is being acquired while laser is being swept. In the case of external K trigger, it can also refer to average acquisition rate.
Δ_{λ}=Lₛ/R (nm/pt)—Spectral resolution
A_{z} (mm)—Axial distance in the tissue over which the OCT scan extends. Axial length of the OCT image. Also know as imaging depth or range, or axial scan range, or scan length

An optical coherence tomography scanner, illustrated in FIG. 1 typically includes a light source, 101. This source can be either a broadband light source with short temporal coherence length or a swept laser source. (See for example, Wojtkowski, et al., “Three-dimensional retinal imaging with high-speed ultrahigh-resolution optical coherence tomography,” Ophthalmology 112(10):1734 2005 or Lee et al. “In vivo optical frequency domain imaging of human retina and choroid,” Optics Express 14(10):4403 2006)

Light from source 101 is routed, typically by optical fiber 105, to illuminate the sample 110, a typical sample being tissues at the back of the human eye. The light is scanned, typically with a scanner 107 between the output of the fiber and the sample, so that the beam of light (dashed line 108) is scanned over the area or volume to be imaged. Light scattered from the sample is collected, typically into the same fiber 105 used to route the light for illumination. Reference light derived from the same source 101 travels a separate path, in this case involving fiber 103 and retro-reflector 104. Those skilled in the art recognize that a transmissive reference path can also be used. Collected sample light is combined with reference light, typically in a fiber coupler 102, to form light interference in a detector 120. The output from the detector is supplied to a processor 130. The results can be stored in the processor or displayed on display 140.

The interference causes the intensity of the interfered light to vary across the spectrum. For any scattering point in the sample, there will be a certain difference in the path length between light from the source and reflected from that point, and light from the source traveling the reference path. The interfered light has an intensity that is relatively high or low depending on whether the path length difference is an even or odd number of half-wavelengths, as these path length differences result in constructive or destructive interference respectively. Thus the intensity of the interfered light varies with wavelength in a way that reveals the path length difference; greater path length difference results in faster variation between constructive and destructive interference across the spectrum. The Fourier transform of the interference spectrum reveals the profile of scattering intensities at different path lengths, and therefore scattering as a function of depth in the sample (see for example Leitgeb et al, “Ultrahigh resolution Fourier domain optical coherence tomography,” Optics Express 12(10):2156 2004). The profile of scattering as a function of depth is called an axial scan (A-scan). A set of A-scans measured at neighboring locations in the sample produces a cross-sectional image (tomogram) of the sample.

The range of wavelengths at which the interference is recorded (spectral range or bandwidth) determines the resolution with which one can determine the depth of the scattering centers, and thus the axial resolution of the tomogram. Recording a limited range of optical frequencies results in a coarser axial resolution.

With this basic framework in mind, various embodiments and aspects of the invention will now be described. The first two embodiments are directed towards swept-source systems while the third embodiment extends the concept to spectral domain OCT systems.

Sweep Rate Adjustment

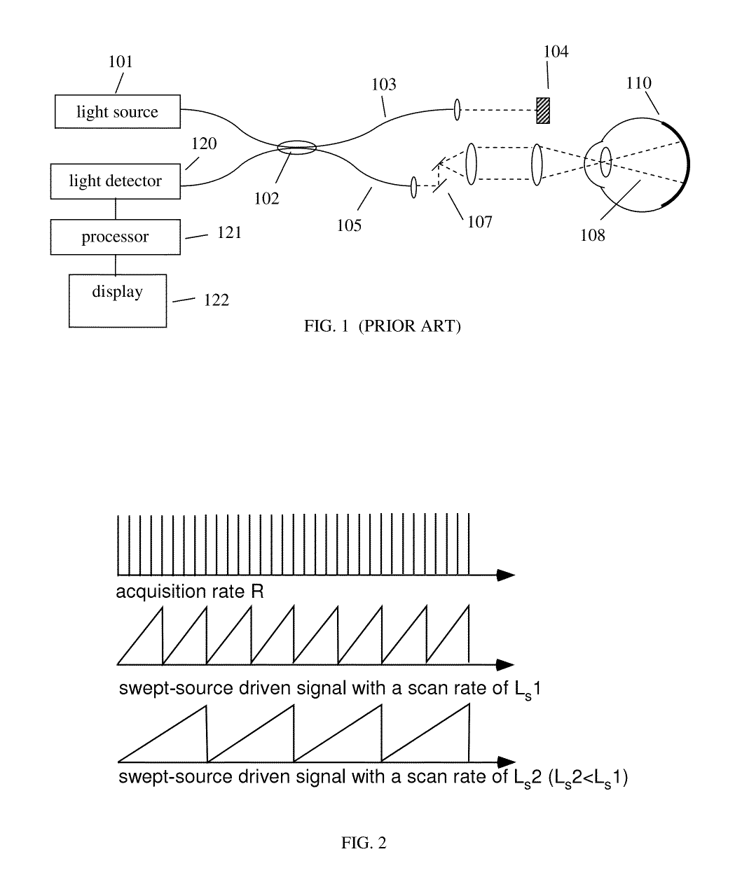

The sweep rate of the swept-source in a SS-OCT system can be changed such that one gets a different scan depth due to improved spectral resolution for the same acquisition rate on the detector, without a need to change the spectral range. The point being that if the data acquisition rate is kept constant and the sweep rate (nm/sec) is adjusted, the spacing between wavelengths detected changes, resulting in an increased scan depth with the slower acquisition, or alternatively a decreased scan depth with a faster acquisition as illustrated schematically in FIG. 2 for the case of two imaging modes. The spectral resolution of the measurements will increase with sampling density unless it is limited by the instantaneous linewidth of the laser. The top line illustrated in the figure illustrates the constant data acquisition rate with data points being collected at even time intervals. The remaining two lines show two different swept-source sweep rates constituting two different imaging modes. In the first imaging mode of this embodiment, the swept-source is driven with a sweep rate Lₛ1. In the second imaging mode the swept-source is driven at a sweep rate of Lₛ2 with Lₛ2<Lₛ1. With the same data acquisition rate of the digitizer (or data acquisition card), the wavelength interval between each data point is decreased for the slower sweep rate, resulting in an increased scan depth in the second imaging mode. If the spectral range (spectral width) of the laser spectrum used in the two imaging modes remains the same, the axial resolution is substantially unchanged.

It must be noted that laser output characteristics such as spectral shape, duty cycle and average power may be affected slightly at different sweep rates. While these changes in laser characteristics may not be significant or substantially modify the axial resolution, there are ways to minimize their impact on the quality of the reconstructed OCT data and hence the axial resolution. Laser characteristics such as spectral shape may be calibrated at different sweep rates and can be corrected by applying suitable spectral shaping functions during the post processing of the OCT signal to minimize the impact on axial resolution at different imaging modes. Additionally, in certain swept-source configurations, it may be possible to provide a feedback to adjust the average optical power output at different sweep rates in order to ensure that the light output power incident on the sample or tissue such as eye does not exceed the safety limit.

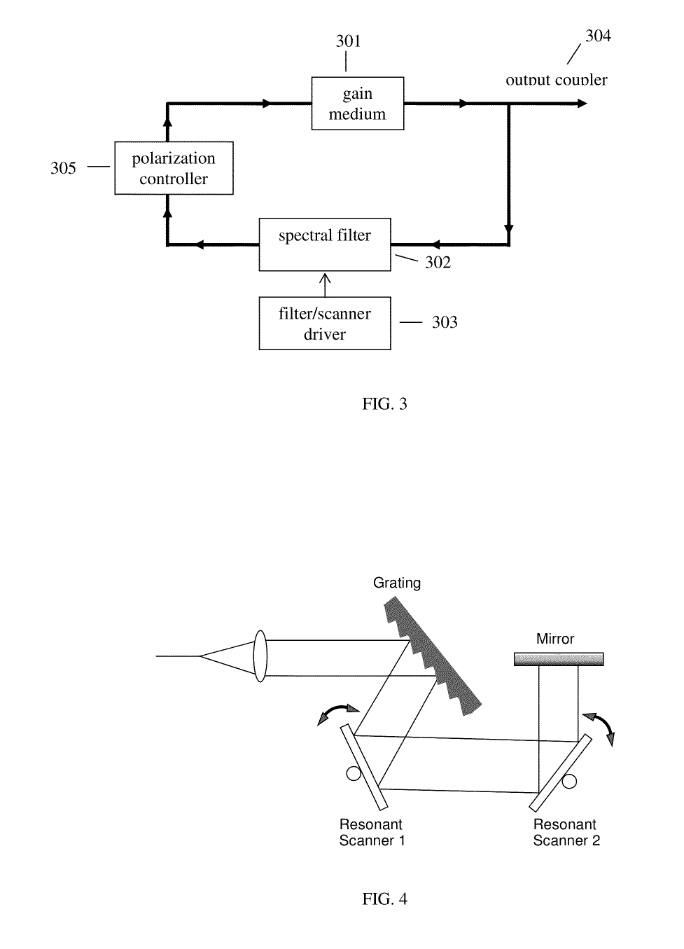

Methods to change the sweep rate of the source can be implemented in various ways and typically involve modifying the signal waveform driving the spectral filter in the swept-source. The effect of changes in laser characteristics as a result of the change in sweep rate may vary from one laser configuration to another. FIG. 3 illustrates the principle of controlling the sweep rates in swept-sources using non-resonant and resonant filtering mechanisms. Typically, the swept-source comprises a gain medium 301 such as a semiconductor optical amplifier (SOA) or a single angle facet (SAF) gain module, a spectral filter element 302, and a scanner/driver arrangement 303 that adjusts the filter to sweep through a series of wavelengths by attenuating undesired wavelengths and keeping desired wavelengths for selective optical amplification. The amplified wavelengths exit the laser cavity at the output coupler 304. A polarization controller 305 is optional for achieving polarization control of the laser. The spectral filter element could be a resonant or non-resonant filter.

While FIG. 3 indicates the use of a single filter and scanner/driver, it is possible to achieve the same effect using multiple filters and drivers. The swept-source device can switch between different resonant filters. The spectral filters can be transmission spectral filters or reflective spectral filters. The multiple scanners of different resonance frequencies (harmonics) could have the same scanning range. Additional designs using the principles described above could be imagined by one skilled in the art.

For swept-sources using resonant scanners or filters, different resonance frequencies of the same scanner can be used. There are many embodiments of adjusting the resonant frequency of a resonant structure included inside the resonant spectral filter. One way of changing the resonant scan rate of scanner is to use different harmonic resonant frequencies of the scanner. The resonant frequency of a resonant structure can be changed by adjusting the mass, inertia, restoring force or other physical parameters of the resonant structure. The resonant frequencies can also be adjusted by the specific design of a spectral filter, such as that implemented in FIG. 4.

FIG. 4 shows the design of one possible embodiment of a reflective spectral filter comprising a diffraction grating, a reflector and multiple scanners. The scanners could either be resonant or non-resonant scanners. The different scanners can run at different scan rates (frequencies) and could have the same (or different) scan range. Using various combinations of the scanners and their respective scan rates, different sweep rates of the laser can be realized.

While the embodiments have focused on the case of two different imaging modes for illustrative purposes, an OCT system can be designed to include an arbitrary number of modes or a variable depth mode depending on the desired applications. The modes could have pre-established imaging depths or the imaging depth could be varied depending on the specific application or portion of the eye being imaged. Specific applications of the invention will be discussed in detail below.

Digitizer Adjustment

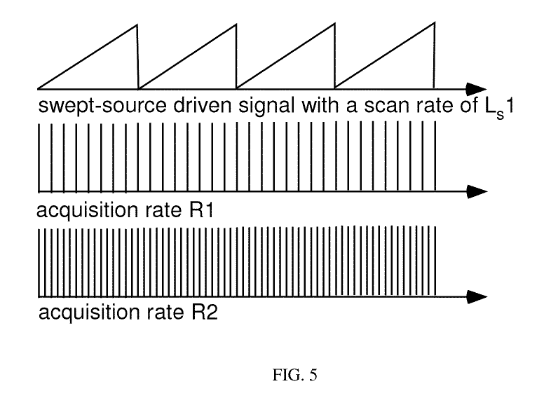

Another way of adjusting the OCT imaging depth in a SS-OCT system without impacting the axial resolution is to adjust the data acquisition rate of the digitizer (data acquisition card) while keeping the swept-source at the same sweep rate. As illustrated in FIG. 5, if the source sweep rate (nm/sec) is kept constant and the data acquisition rate is increased, the spacing between wavelengths detected decreases, resulting in an increased scan depth with a denser acquisition, or alternatively a decreased scan depth with a sparser acquisition. While it may not be desirable to operate an optimized data acquisition system at a lower acquisition rate, this embodiment has the advantage that the sweep rate remains unchanged and hence the spectral characteristics of the source remain the same across various imaging depth modes eliminating any impact on axial resolution.

For example, with a 100 KHz swept-source of 50% duty cycle, a digitizer of 200 M (Samples/sec) acquisition rate records 1024 wavelength samples within 5 micro-seconds. An increased acquisition rate of 400 M (Samples/sec) within the same 5 micro-seconds (covering the same wavelength range of the swept-source) doubles the acquisition samples and the imaging depth.

If the digitizer is running in an “external clock” mode, it takes external K-clock signals generated by the laser or OCT system as the sampling clock signal. Fabry-Pérot interferometer (or etalon) or Mach-Zehnder interferometer (MZI) are normally used to generate K-clock signals in swept-source designs. These K-clock signals can be tuned to variable rates by adjusting the delay in the MZI or the separation/refractive index of the etalon. The delay in the K-clock interferometer is proportional to the maximum depth of the system. A specific embodiment of the invention related to K-clock based data acquisition systems will be described in detail below.

As the data acquisition is changed, the detector bandwidth should also be adjusted to achieve the optimal system performance and sensitivity. The optimal bandwidth of the detector should be set approximately to half the acquisition rate of the digitizer (Nyquist bandwidth criterion).

Frequency Multiplier/Divider

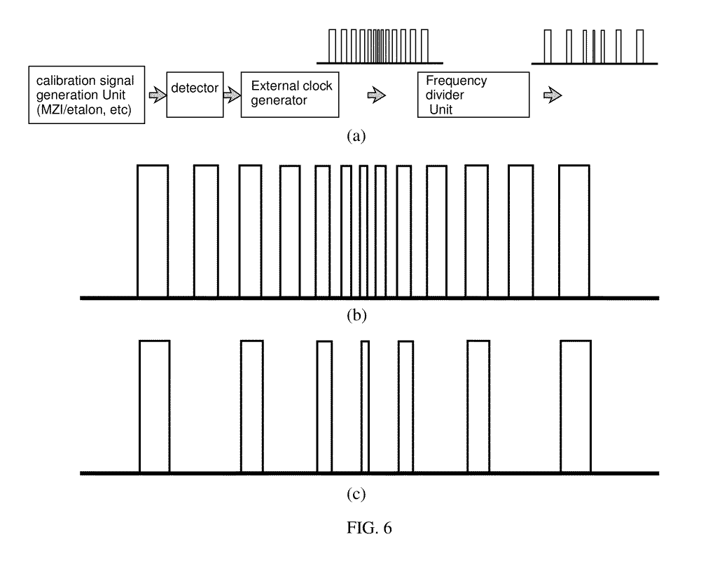

FIG. 6 shows a schematic of K-clock generation in SS-OCT with variable frequencies. As described above, in some embodiments of SS-OCT systems, the calibration signal from a MZI or etalon is picked up by a detector. The electronic signal from the detector is input to an external clock generator to generate a generic external clock.

The physical parameters of the calibration signal generation unit (such as the optical delay in the MZI or FSR of the etalon) are selected such that a dense external clock is generated to achieve a longer imaging depth in the SS-OCT system, such as 6 mm in tissue. In the first imaging mode, the initially generated external clock is directly used for data acquisition in the SS-OCT system, or the Frequency Divider Unit (FDU) is running in a mode such that the external clock signal after the FDU is still dense enough to get a 6 mm imaging depth in tissue.

In the second imaging mode or mode of operation, a FDU reduces the initial external clock (FIG. 6(b)) to generate a second external clock of lower frequency (such as FIG. 6(c)), which is then used for data acquisition for shorter imaging depth in the SS-OCT system. In practice, the clock duty cycle in FIG. 6(c) can be optimized to ˜50% to optimize the overall performance of the analog-to-digital convertor and the data acquisition in SS-OCT. The symmetry present in the K-clock signal shown in the figure was chosen to have the densest scanning in the center of the spectral range where the instantaneous tuning rate may be higher, but the inventive method will apply to any external clock pattern.

Note that in this embodiment, the physical parameters of the calibration signal generation unit (such as the optical delay in the MZI or FSR of the etalon) are not physically adjusted for variable imaging depth.

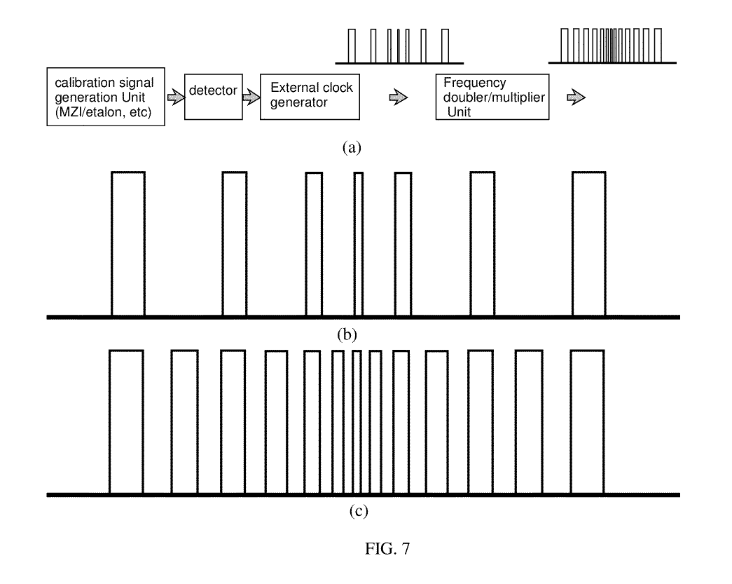

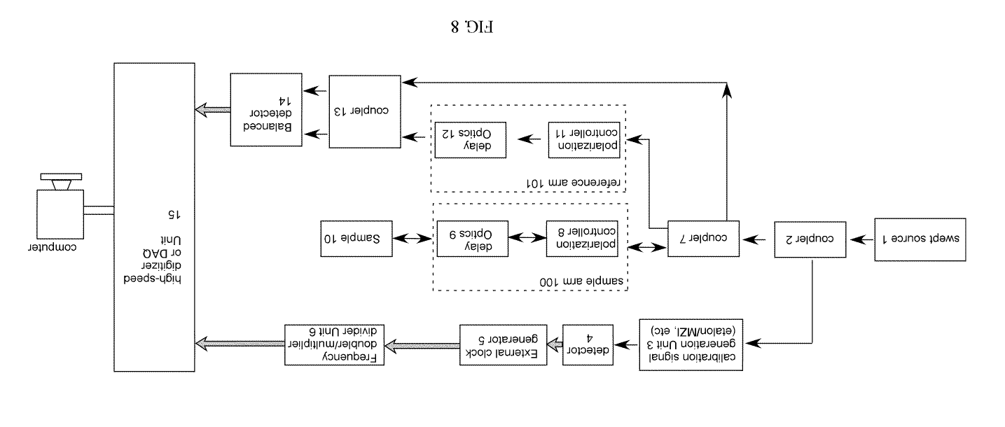

In another embodiment illustrated in FIG. 7, the optical delay in the MZI or etalon is selected such that a sparse external clock (FIG. 7(b)) is generated to achieve a shorter imaging depth in the SS-OCT system, such as 2 mm in tissue. The generated external clock is used for data acquisition in the first imaging mode in the SS-OCT system.

In the second imaging mode or mode of operation, the generated external clock is input into a Frequency Doubler/Multiplier Unit to generate a second external clock of higher frequency (FIG. 7(c)), which is then used for data acquisition for longer imaging depth in the SS-OCT system. Again, the optical delay of the MZI or etalon is not physically changed for variable imaging depth in this embodiment.

FIG. 8 shows a schematic of a SS-OCT design with an external clock. The light from the swept-source 1 is coupled into coupler 2, which splits part of the light going to a calibration signal generation unit 3, such as a MZI or an etalon. The calibration signal is captured by a detector 4 and then goes to external clock generator 5 to generate an external clock. The Frequency Doubler/Multiplier/Divider Unit 6 takes the initial external clock as an input and generates an external clock of different frequencies, depending on the setting of unit 6. The majority of the light from the swept-source 1 is delivered to the sample arm 100 and reference arm 101 through coupler 2 and coupler 7. The reference arm 101 is composed of a polarization controller 11 and delay optics 12. In the sample arm, the light passes through the polarization controller 8, the delay optics 9 and hits the sample 10. The light reflected by the sample 10 passes once again through the delay optics 9 and polarization controller 8 and coupler 7, which sends part of the light to coupler 13 (normally a 50/50 coupler), which combines 50% of light from the reference path and 50% from the sample path. The OCT interference signal is then detected by a balanced detector 14 and digitized by a high-speed digitizer or DAQ Unit 15.

As in the case of changing sweep rate, while the embodiments discussed above for adjusting the data acquisition rate describe the use of two imaging modes, there could be any arbitrary number of imaging modes or the imaging mode could be variable depending on input from the user or adjusted automatically by the system depending on factors such as the specific area of tissue to be imaged. Additionally both the data acquisition rate and the laser sweep rate could be adjusted in combination to achieve a desired imaging depth.

SD-OCT Embodiment

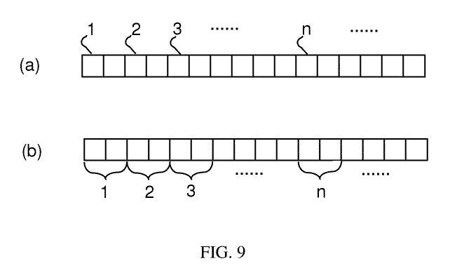

Until this point, focus has been placed on SS-OCT systems. In a SD-OCT system, a similar embodiment of the present invention illustrated in FIG. 9 is to take (or discard) signals from every other detector element or pixel of the spectrometer (FIG. 9(a)) or laterally bin signals of two or more adjacent detector elements (FIG. 9(b)). The covered spectral range in the spectrometer can be the same. The spacing between valid sampled wavelengths increases, resulting in a decreased scan depth. The frame rate can be increased due to the fact that the data points to be digitized and transferred to the processing unit are reduced. Furthermore, if binning is used, the total number of photons contributing to one data point increases, compensating for the reduction in signal-to-noise ratio due to the shorter integration time with increased frame rate.

Ophthalmic Applications of Variable Imaging Depth

As will be described below, there are many applications where one might want to adjust the imaging or scan depth of an OCT instrument while acquiring data, or provide multiple options for different depth ranges by changing either the sweep rate or the data acquisition rate without substantially changing the spectral bandwidth and therefore the axial resolution of the system. The OCT could be part of an ophthalmic instrument, or a different device. Changing the OCT imaging depth by changing the laser sweep rate (nm/s) is of particular interest as it changes the number of A-scans/sec acquired for a given spectral range of the laser. One can either decrease the depth range to increase the A-scan rate, or decrease the A-scan rate in order to increase the depth range.

Variable imaging depth methods can provide different imaging ranges for imaging different portions of the eye—retina, anterior chamber, eye length, choroid, cornea, optic disc, vitreous region, lens, periphery etc. The assumption is that the smallest range necessary to acquire useful data should be used to maximize the number of A-scans/sec. For some locations in the eye, the structure of interest is thicker than for other locations in the eye (e.g. anterior chamber requires 6 mm while retina typically requires only 2 mm). In addition, for some structures, a larger axial range gives flexibility to the user in setting up the scan in a way that optimizes the resulting image. An example is angle imaging, where a scan deeper than 2 mm can help the user set up the scan (e.g. by adjusting the patient fixation direction) to get the cornea to appear flat, ensuring a good view of horizontal structures such as the scleral spur as well as reducing the distorting effects of refraction. Another example is the optic disc, where the structures of interest typically fit within a 2 mm axial range, but any patient motion during the scan may cause the tissue to leave that range, reducing the usefulness of the acquired data. A further example is the periphery of the eye, where the tilt may make it difficult to capture the thin retina in a 2 mm deep scan.

Variable imaging depth provides the ability to adjust scan depth to account for differences between tissue structures in different people's eyes, for instance the ability to increase the scan range to account for a swollen retina, or to account for myopia where the axial location of the tissue changes quickly with transverse position in the retina. Such an adjustment could either be made automatically based on information from a previous visit or knowledge of the state of the eye, or could be made during scanning in response to either the operator or an algorithm that detects one of these situations.

Variable imaging depth provides the ability for the operator to either select an imaging range prior to starting a scan, or adjust the imaging range as necessary during the scan. The scan range options could either be a smoothly varying scale, or a limited set of range choices.

Variable imaging depth allows for the possibility of one scan range for patient alignment purposes, followed by a second shorter scan range for acquisition. Here one would identify a surface of interest (front of eye or retina as examples) with the first scan range, adjust the axial scan position such that the tissue of interest is near the center of the scan range, then reduce the scan range for the purpose of acquiring an image of the tissue of interest.

Variable imaging depth allows for the acquisition of a sequence of images (two or more) with different scan depths on the same eye, for instance first acquiring an image of the anterior chamber with roughly a 6 mm in tissue scan depth, followed by an image of the retina with roughly a 2 or 3 mm in tissue scan depth. The sequence of images could also be used to correct certain undesired features (e.g. due to the mirror image) that are present in one image but not the other or guide the quantitative analysis in one image by correlating it with features in the other image.

The scan depth can be varied in conjunction with changing the size of the OCT beam on the pupil. Increasing the beam size on the pupil provides a smaller spot size on the retina and reduces the depth of focus. The reduced depth of focus alleviates the need for a long scan depth, while the smaller spot makes it desirable to acquire more transverse points. Increasing the sweep rate of the laser both reduces the scan length, and increases the rate at which A-scans are collected making it possible to acquire more transverse points in a given time. Therefore, this application would involve providing two or more scan options, where one scan option has both an increased OCT beam size on the pupil and an increased sweep rate of the laser relative to another scan.

In uveitis, there may be inflammation, haze, glare or cells in regions of the eye that are not typically imaged with OCT. A scan that could sample the eye from the back of the iris and/or lens to the retina might be able to detect, quantify, and record inflammations that currently requires subjective grading at the slit lamp.

It might be useful to use a large imaging depth scan (such as one that might be used to determine axial length) over a broad range with sparse transverse sampling to create a template of the curvature of the back of the eye. Such a template could be used to set the axial positions of subsequent retinal A-scans (acquired with less axial depth and a higher A-scan rate) to keep the tissue of interest within the scan range during transverse scanning. Currently OCT scans show variable tilt and curvature in the OCT data that depend on how the operator took the scan. Variable depth scanning could allow the true geometry of the back of the eye to be determined, and use this information to optimize later scanning.

Combining the techniques described herein for adjusting imaging depth by changing the sweep speed and/or data acquisition speed with full-range OCT imaging techniques provides further opportunities for imaging depth variability without impacting the axial resolution. These techniques could be extremely useful for biomedical imaging applications such as anterior segment imaging of the eye, since the useful imaging range could be doubled. In FD-OCT the real-valued spectral interferogram is Fourier transformed (after some data processing steps such as background subtraction, dispersion compensation etc.) and the resulting image in full-range OCT contains the complex conjugate artifact (or ‘mirror image’) that minors the true image about the zero optical path delay point. The artifact can result in ambiguity in image interpretation and has in practice required that the location of the zero-delay location be limited within the sample to avoid the overlap, effectively halving the potential imaging depth. It is highly desirable to be able to double the depth imaging range of OCT while minimizing the full-range OCT imaging related artifacts such as the mirror image. There are a range of hardware and post-processing methods that can be used to obtain optimized full-range OCT images including for example stepping phase shifting in the reference arm using piezo-mounted reference minors, electro-optic modulators, carrier-frequency shifting methods, quadrature interferometers, and polarization diversity. Additionally there are some software based approaches such as dispersion encoded full-range (DEFR) algorithm that utilizes the dispersion mismatch between sample and reference arm to iteratively suppress complex conjugate artifacts. While we have discussed some of the full-range OCT techniques, those skilled in the art can imagine other methods to implement it.

Although various applications and embodiments that incorporate the teachings of the present invention have been shown and described in detail herein, those skilled in the art can readily devise other varied embodiments that still incorporate these teachings.

The following references are hereby incorporated by reference:

### PATENT DOCUMENTS

US Publication No. 2007/0024856 Izatt et al “Optical coherence imaging systems having a reduced effective linewidth and methods of using the same”

US Publication No. 2010/0110376 Everett et al “Variable resolution optical coherence tomography scanner and method for using same”

Publication No. WO 2010/006785 Hacker et al “Optical coherence tomography methods and systems”

Publication No. WO 2011/037980 Buckland et al “Systems for extended depth frequency domain optical coherence tomography (FDOCT) and related methods”

### NON-PATENT LITERATURE

M. Gora et al “Ultra high-speed swept-source OCT imaging of the anterior segment of human eye at 200 kHz with adjustable imaging range,” Optics Express 17(17):14880 2009.

J. Zhang et al “Swept laser source at 1 um for Fourier domain optical coherence tomography,” Applied Physics Letters 89, 073901 2006.

J. Xi et al “Generic real-time uniform K-space sampling method for high-speed swept-source optical coherence tomography,” Optics Express 18(9):9511 2010.

Wojtkowski, et al., “Three-dimensional retinal imaging with high-speed ultrahigh-resolution optical coherence tomography,” Ophthalmology 112(10):1734 2005.

Lee et al. “In vivo optical frequency domain imaging of human retina and choroid,” Optics Express 14(10):4403 2006.

Leitgeb et al, “Ultrahigh resolution Fourier domain optical coherence tomography,” Optics Express 12(10):2156 2004.

What is claimed is:

1. A swept-source optical coherence tomography (SS-OCT) system generating images of the eye comprising:
a light source for generating a probe beam wherein said source is swept over a spectral range at a sweep rate;
optics for scanning the beam over a set of transverse locations across the eye;
a detector for measuring light returned from the eye as a function of wavelength that acquires data at a data acquisition rate; and
a processor for generating images of the eye based on the output of the detector over a sampling of wavelengths, said SS-OCT system capable of switching between imaging modes with different imaging depths by doing one or both of adjusting the sweep rate of the source or the data acquisition rate of the detector while the source is swept over substantially the same spectral range at each transverse location

2. A system as recited in claim 1, wherein the different imaging modes are used to image different portions of the eye.

3. A system as recited in claim 2, wherein the portions of the eye are selected from the retina, the anterior chamber, the choroid, the cornea, lens, vitreous region, and the optic disc.

4. A system as recited in claim 1, wherein the data acquisition rate is adjusted using a frequency multiplier or divider unit.

5. A system as recited in claim 1, wherein the light source is swept over the spectral bandwidth by means of a resonant spectral filter.

6. A system as recited in claim 1, wherein the light source is swept over the spectral bandwidth by means of a non-resonant spectral filter.

7. A system as recited in claim 1, wherein the light source is swept over the spectral range using multiple spectral filters.

8. A system as recited in claim 1, wherein the switch between imaging modes is implemented based on input from the system operator.

9. A system as recited in claim 1, wherein the switch between imaging modes is made automatically by the instrument.

10. A system as recited in claim 1, further comprising means to adjust the size of the OCT beam on the pupil in conjunction with the switching between imaging modes.

11. A system as recited in claim 1, wherein the imaging depths of the imaging modes are variable.

12. A system as recited in claim 1, further comprising means to reduce the complex conjugate artifact while obtaining a full-range OCT image.

13. A system as recited in claim 1, further comprising means to correct for any change in spectral properties of the probe beam resulting from adjusting the sweep rate.

14. A system as recited in claim 1, wherein the data acquisition rate is controlled by an external clock.

15. A swept source optical coherence tomography (OCT) system comprising:
a light source for generating a beam of radiation;
a driver associated with the light source arranged to sweep the wavelength of the light source over a predetermined spectral range and at a particular sweep rate;
a beam divider for separating the beam of radiation into a sample arm and a reference arm;
optics for scanning the beam in the sample arm over a set of transverse locations on a sample;
a detector for measuring radiation returning from both the sample arm and the reference arm, the detector generating output signals at an acquisition rate; and
a processor for converting the output signals into image information, said processor further controlling the sweep rate of the driver and the acquisition rate of the detector in a manner to change the imaging depth while utilizing said predetermined spectral range for generating the images so that the axial image resolution will remain substantially constant.

16. A system as recited in claim 15 wherein said processor functions to reduce the sweep rate in order to increase the imaging depth and increase the sweep rate in order to decrease the imaging depth.

17. A system as recited in claim 15 wherein said processor functions to increase the acquisition rate in order to increase the imaging depth and decrease the acquisition rate in order to decrease the imaging depth.

18. A system as recited in claim 15, wherein the acquisition rate is adjusted using a frequency multiplier or divider unit.

19. A system as recited in claim 15, wherein the driver includes one of a resonant or non-resonant spectral filter.

20. A system as recited in claim 15, further comprising means to correct for any change in spectral properties of the radiation resulting from adjusting the sweep rate.

21. A system as recited in claim 15, wherein the sample is a human eye and imaging depth is adjusted to image different portions of the eye.

22. A system as recited in claim 21, wherein the portions of the eye are selected from the retina, the anterior chamber, the choroid, the cornea, lens, vitreous region, and the optic disc.

23. A system as recited in claim 15, wherein the imaging depth is changed during the course of a single scan.
